(19) United States
(12) Patent Application Publication
(10) Pub. No.: US 2009/0156645 A1
(43) Pub. Date: Jun. 18, 2009

(54) Method for Increasing the Susceptibility of Peptide Deformylase Inhibitors by Using Efflux Pump Inhibitors

(76) Inventors: Charles Richard Dean, Nahant, MA (US); Neil Stewart Ryder, Concord, MA (US)

Correspondence Address: LAHIVE & COCKFIELD, LLP;FLOOR 30, SUITE 3000, ONE POST OFFICE SQUARE, BOSTON, MA, 02109, US

(21) Appl. No.: 11/570,954

(22) PCT Filed: Jun. 29, 2005
(86) PCT No.: PCT/EP2005/007008

§ 371 Date: Oct. 6, 2008

Related U.S. Application Data
(60) Provisional application No. 60/584,023, filed on Jun. 30, 2004.

(51) Int. Cl. A61K 31/4439; A61P 31/04

(52) U.S. Cl. 514/343

(57) ABSTRACT

The present invention provides methods and compositions for increasing the susceptibility of PDF inhibitors against Gram-negative organisms by using efflux pump inhibitors.

### FIELD OF THE INVENTION

The present invention relates to peptidyl deformylase (PDF) inhibitors and in particular, to methods for improving the effectiveness of PDF inhibitors against Gram-negative bacteria by utilizing efflux pump inhibitors.

### BACKGROUND OF THE INVENTION

The continued emergence and spread of target-based resistance to the current armamentarium of antibiotics makes obvious the need for either compound modifications aimed at circumvention of these mechanisms, or better, the discovery of compounds directed at novel cellular functions for which pre-existing target-based resistance would not be expected to confer cross-resistance. A hovel class of hydroxylamine compounds inhibiting the so-far unexploited function of PDF show great promise in this regard, particularly towards Gram-positive bacteria, including those with well-characterized resistance mechanisms for commonly used antibiotics. See WO 02/102790A1. PDF is a metallopeptidase found in prokaryotic organisms such as bacteria. Protein synthesis in prokaryotic organisms begins with N-formyl methionine (fMet). After initiation of protein synthesis, the formyl group is removed by PDF. This activity is essential for maturation of proteins. It has been shown that PDF is required for bacterial growth. Since synthesis in eukaryotic organisms does not depend on fMet for initiation, agents that will inhibit PDF are attractive candidates for development of new antimicrobial and antibacterial drugs.

A major potential hurdle in the development of these novel agents however are those general mechanisms responsible for intrinsic resistance to a wide range of structurally unrelated compounds. These include membrane impermeability and efflux, which manifest most acutely in the case of Gram-negative bacteria, where the outer membrane and efflux pumps have been shown to act synergistically to minimize intracellular accumulation of a variety of structurally unrelated compounds. See Nikaido, J Bacteriol, Vol. 178, No. 20, pp. 5853-5859 (1996); Nikaido, Curr Opin Microbiol, Vol. 1, No. 5, pp. 516-523 (1998); and Nikaido and Zgurskaya, J Mol Microbiol Biotechnol, Vol. 3, No. 2, pp. 215-218 (2001). The Gram-negative efflux pumps of the resistance-nodulation-cell division (RND) family appear to have the broadest substrate range, and are therefore most generally relevant vis a vis drug resistance in Gram-negatives. Architecturally, they consist of an inner membrane proton-drug antiporter, an outer membrane channel and a so-called membrane fusion protein that is thought to function in facilitating the interaction between the inner- and outer-membrane components in the periplasm. Substrate extrusion is driven by the proton motive force and recent data indicates that substrates may be pumped from the periplasm or the inner leaflet of the cytoplasmic membrane. See Elkins and Nikaido, J Bacteriol, Vol. 184, No. 23, pp. 6490-6498 (2002); Li, Ma, Livermore and Nikaido, Antimicrob Agents Chemother, Vol. 38, No. 8, pp. 1742-1752 (1994); and Yu et al., Science, Vol. 300, No. 5621, pp. 976-980 (2003).

The broad substrate range accommodated by efflux pumps, particularly those of the RND family, is such that their potential to render less effective even highly target-active novel antimicrobials must be anticipated and considered in drug development programs striving for broad-range or Gram-negative coverage. Moreover, regulatory mutations turning on or increasing efflux pump expression, presumably selected for by exposure to antimicrobial agents or biocides, can confer additional resistance to several or all of the substrates for a given pump. See Chuanchuen et al., Antimicrob Agents Chemother, Vol. 45, No. 2, pp. 428-432 (2001). Efflux pump overexpressors have been isolated clinically [see Beinlich, Chuanchuen and Schweizer, FEMS Microbiol Lett, Vol. 198, No. 2, pp. 129-134 (2001); Mazzariol et al., Antimicrob Agents Chemother, Vol. 44, No. 12, pp. 3441-3443 (2000); and Ziha-Zarifi et al., Antimicrob Agents Chemother, Vol. 43, No. 2, pp. 287-291 (1999)], and pump over-expression can contribute significantly to increased drug resistance. Therefore, while cross resistance to novel agents may not pre-exist in the form of target based mutations selected by other antibiotics, antimicrobial exposures may select pump mutants with decreased susceptibility to new agents.

Pseudomonas aeruginosa (P. aeruginosa), an important emerging opportunistic pathogen, represents one end of the spectrum of efflux based resistance, having multiple RND family pumps of overlapping substrate range and a notably impermeable outer membrane which has been shown to significantly increase the efficiency of the pumps by limiting influx. See Poole, J Mol Microbiol Biotechnol, Vol. 3, No. 2, pp. 255-264 (2001). Perhaps representing the other end is Haemophilus influenzae (H. influenzae), an important respiratory pathogen [see Dagan et al., Pediatr Infect Dis J, Vol. 19, No. 2, pp. 95-104 (2000); Gotfried, Am J Med, Vol. 111, Suppl. 9A, pp. 25S-29S (2001); Pfaller, Ehrhardt and Jones, Am J Med, Vol. 111, Suppl. 9A, pp. 4S-12S (2001); and Sokol, Am J Med, Vol. 111, Suppl. 9A, pp. 19S-24S (2001)] that has only one known RND family (acrAB homolog) pump [see Fleischmann et al., Science, Vol. 269, No. 5223, pp. 496-512 (1995); and Sanchez, Pan, Vinas and Nikaido, J Bacteriol, Vol. 179, No. 21, pp. 6855-6857 (1997)] and is characterized by a relatively permeable outer membrane. The increased permeability of the outer membrane has been implicated in limiting the efficiency of the efflux pump even for relatively large substrates such as erythromycin. See Sanchez, Pan, Vinas and Nikaido (1997), supra. Therefore, H. influenzae may represent an example of a Gram-negative pathogen where efflux based intrinsic and acquired resistance may be expected to pose less of a problem. Despite this, and consistent with erythromycin being a substrate of the acrAB homolog of H. influenzae [see Sanchez, Pan, Vinas and Nikaido (1997), supra], moderate levels of intrinsic resistance to macrolides in H. influenzae clinical isolates has been associated with efflux. See Peric, Bozdogan, Jacobs and Appelbaum, Antimicrob Agents Chemother, Vol. 47, No. 3, pp. 1017-1022 (2003). It has also been observed that H. influenzae clinical strains show generally reduced susceptibility to various novel PDF inhibitors.

In summary, the above discussion indicates that the active efflux of antibacterial agents by efflux pumps present in bacteria, particularly Gram-negative bacteria, can be an important factor in contributing to resistance of the bacteria to these agents. Accordingly, the development and use of inhibitors of bacterial efflux pumps in combination with antibacterial agents may improve the susceptibility of bacteria, particularly Gram-negative bacteria, to various antibacterial agents for which the bacteria have reduced susceptibility due to the agents' efflux from the bacteria.

### SUMMARY OF THE INVENTION

In one aspect, the present invention provides a method for treating a Gram-negative bacterial infection in a mammal is provided, which comprises administering an effective amount of a compound of formula (I)

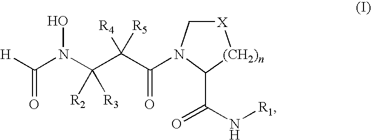

wherein
X is —CH₂—, —S—, —CH(OH), —CH(OR)—, —CH(SH)—, —CH(SR)—, —CF₂—, —C═N(OR)— or —CH(F)—, wherein R is alkyl;
R₁ is aryl or heteroaryl;
each of R₂, R₃, R₄ and R₅, independently, is hydrogen or alkyl, or
R₂ or R₃ and R₄ or R₅, collectively, form a C₄₋₇cycloalkyl; and
n is 0-3, provided that when n is 0, X is —CH₂—,
   or a salt thereof or a prodrug thereof;
   and an efflux pump inhibitor.

In one useful embodiment, the present invention provides a method for treating a Gram-negative bacterial infection in a mammal which is caused by a Gram-negative bacteria possessing an RND efflux pump. The method comprises administering an effective amount of the compound

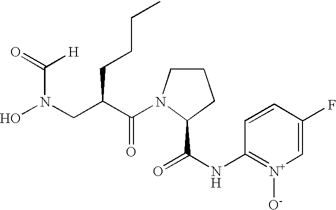

and an efflux inhibitor that inhibits the RND efflux pump in the Gram-negative bacteria.

In another aspect, the present invention provides a method for increasing the susceptibility of Gram-negative bacteria to a compound of formula (I):

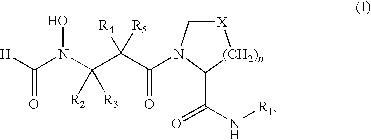

wherein
X is —CH₂—, —S—, —CH(OH)—, —CH(OR)—, —CH(SH)—, —CH(SR)—, —CF₂—, —C═N(OR)— or —CH(F)—, wherein R is alkyl;
R₁ is aryl or heteroaryl;
each of R₂, R₃, R₄ and R₅, independently, is hydrogen or alkyl, or
R₂ or R₃ and R₄ or R₅ collectively form a C₄₋₇cycloalkyl; and
n is 0-3, provided that when n is 0, X is —CH₂—; or
   a salt thereof or a prodrug thereof. The method comprises contacting the bacteria with the compound of formula (I) and an efflux pump inhibitor in an amount effective to inhibit an efflux pump in the Gram-negative bacteria.

In one embodiment, the present invention provides a method for increasing the susceptibility of Gram-negative bacteria possessing an RND efflux pump to the compound

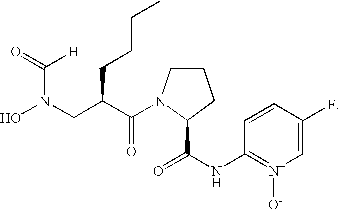

The method comprises contacting the Gram-negative bacteria with the compound and an efflux pump inhibitor in an amount effective to inhibit the RND pump in the Gram-negative bacteria.

In another aspect, the present invention provides a pharmaceutical composition comprising a compound of formula (I)

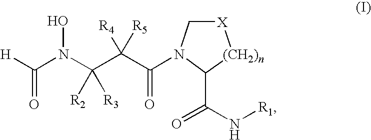

wherein
X is —CH₂—, —S—, —CH(OH)—, —CH(OR)—, —CH(SH)—, —CH(SR)—, —CF₂—, —C═N(OR)— or —CH(F)—, wherein R is alkyl;
R₁ is aryl or heteroaryl;
each of R₂, R₃, R₄ and R₅, independently, is hydrogen or alkyl, or
R₂ or R₃ and R₄ or R₅, collectively, form a C₄₋₇cycloalkyl; and
N is 0-3, provided that when n is 0, x is —CH₂—,
   or a salt thereof or a prodrug thereof, an efflux pump inhibitor and a pharmaceutically acceptable carrier.

### BRIEF DESCRIPTION OF THE DRAWINGS

FIG. 1. Structures of the novel PDF inhibitors LBM415 and LBK611.

FIG. 2. Genetic arrangement of the acrAB efflux pump genes in H. influenzae showing the positions and orientations of kanamycin resistance markers (arrows) used for insertional inactivation. Also shown is the position and orientation of the kanamycin resistance marker inserted in ORF HI1462 (toIC).

FIG. 3. Region of acrA missing in clinical isolate NB65062 (black bar). The positions of primers used for PCR are indicated. Primer pair acrAHIF/acrAHIR do not generate a product since the site for acrAHIF is missing in NB65062.

FIG. 4. Comparison of truncated acrR genes from four clinical H. influenzae isolates with reduced susceptibility to LBM415 and LBK611, to wild-type acrR. The mutations are as follows: NB65016, 1 base (C) insertion after nucleotide 442 (frameshift); NB65027, 8 bp deletion-GTT insertion after nucleotide 366 (frameshift), additional 1 base insertion downstream; NB65051, 4 bp deletion after nucleotide 322 (frameshift); NB65063, C252T substitution (stop).

### DETAILED DESCRIPTION OF THE INVENTION

All patent applications, patents and literature references cited herein are hereby incorporated by reference in their entirety.

Unless otherwise stated, the following terms as used in the specification have the following meaning.

The term “cycloalkane” or “cycloalkyl” contains from 3- to 7-ring carbon atoms, and is, e.g., cyclopropyl, cyclobutyl, cyclopentyl and cyclohexyl.

The term “alkyl” refers to saturated or unsaturated aliphatic groups, such as alkenyl or alkynyl, cycloalkyl or substituted alkyl including straight-chain, branched-chain and cyclic groups having from 1-10 carbons atoms. Preferably “alkyl” or “alk”, whenever it occurs, is a saturated aliphatic group or cycloalkyl, more preferably C₁₋₇alkyl, particularly C₁₋₄alkyl. Examples of “alkyl” or “alk” include, but are not limited to, methyl, ethyl, n-propyl, isopropyl, n-butyl, isobutyl, sec-butyl, t-butyl, n-pentyl, neopentyl, n-hexyl or n-heptyl, cyclopropyl and, especially, n-butyl.

The term “substituted alkyl” refers to an alkyl group that is substituted with 1 or more substituents, preferably 1-3 substituents including, but not limited to, substituents, such as halogen, lower alkoxy, hydroxy, mercapto, carboxy, cycloalkyl, aryl, heteroaryl and the like. Examples of substituted alkyl groups include, but are not limited to, —CF₃, —CF₂—CF₃, hydroxymethyl, 1- or 2-hydroxyethyl, methoxymethyl, 1- or 2-ethoxyethyl, carboxymethyl, 1- or 2-carboxyethyl and the like.

The term “aryl” or “Ar” refers to an aromatic carbocyclic group of 6-14 carbon atoms having a single ring including, but not limited to, groups, such as phenyl; or multiple condensed rings including, but not limited to, groups, such as naphthyl or anthryl; and is especially phenyl.

The term “heteroaryl” or “HetAr” refers to a 4- to 7-membered, monocyclic aromatic heterocycle or a bicycle that is composed of a 4- to 7-membered, monocyclic aromatic heterocycle and a fused-on benzene ring. The heteroaryl has at least 1 hetero atom, preferably 1 or 2 heteroatoms including, but not limited to, heteroatoms, such as N, O and S, within the ring. A preferred heteroaryl group is pyridinyl, pyrimidinyl or benzodioxolanyl.

The aryl or heteroaryl may be unsubstituted or substituted by 1 or more substituents including, but not limited to, C₁₋₇alkyl, particularly C₁₋₄alkyl, such as methyl, hydroxy, alkoxy, acyl, acyloxy, SCN, halogen, cyano, nitro, thioalkoxy, phenyl, heteroalkylaryl, alkylsulfonyl and formyl.

The term “carbonylamine”, as used herein, refers to a —NHC(O)— group, wherein the amino portion of the group is linked to the aryl/heteroaryl and the carbonyl portion of the group is linked to the azacyclo₄₋₇alkane, thiazacyclo₄₋₇alkane or imidazacyclo₄₋₇alkane.

The term “heteroalkyl” refers to saturated or YY unsaturated C₁₋₁₀alkyl as defined above, and especially C₁₋₄heteroalkyl which contain 1 or more heteroatoms, as part of the main, branched or cyclic chains in the group. Heteroatoms may independently be selected from the group consisting of —NR—, where R is hydrogen or alkyl, —S—, —O— and —P—; preferably —NR—, where R is hydrogen or alkyl; and/or —O—. Heteroalkyl groups may be attached to the remainder of the molecule either at a heteroatom (if a valence is available) or at a carbon atom. Examples of heteroalkyl groups include, but are not limited to, groups, such as —O—CH₃, —CH₂—O—CH₃, —CH₂—CH₂—O—CH₃, —S—CH₂—CH₂—CH₃, —CH₂—CH(CH₃)—S—CH₃ and —CH₂—CH₂—NH—CH₂—CH₂—.

The heteroalkyl group may be unsubstituted or substituted with 1 or more substituents, preferably 1-3 substituents including, but not limited to, alkyl, halogen, alkoxy, hydroxyl, mercapto, carboxy and, especially, phenyl. The heteroatom(s), as well as the carbon atoms of the group may be substituted. The heteroatom(s) may also be in oxidized form.

The term “alkoxy”, as used herein, refers to a C₁₋₁₀alkyl linked to an oxygen atom or, preferably C₁₋₇alkoxy, more preferably C₁₋₄alkoxy. Examples of alkoxy groups include, but are not limited to, groups, such as methoxy, ethoxy, n-butoxy, tert-butoxy and allyloxy.

The term “acyl”, as used herein, refers to the group —(O)CR, where R is alkyl, especially C₁₋₇alkyl, such as methyl. Examples of acyl groups include, but are not limited to, acetyl, propanoyl and butanoyl.

The term “acyloxy”, as used herein, refers to the group —OC(O)R, wherein R is hydrogen or alkyl, especially C₁₋₇alkyl, such as methyl or ethyl; or phenyl or substituted alkyl, as defined above.

The term “alkoxycarbonyl”, as used herein, refers to the group —COOR, wherein R is alkyl, especially C₁₋₇alkyl, such as methyl or ethyl.

The term “halogen” or “halo”, as used herein, refers to chlorine, bromine, fluorine, iodine and, is especially, fluorine.

The term “thioalkoxy”, as used herein, means a group —SR, where R is an alkyl, as defined above, e.g., methylthio, ethylthio, propylthio, butylthio and the like.

The term “heteroalkylaryl”, as used herein, means a heteroalkyl group, e.g., —O—CH₂— substituted with an aryl group, especially phenyl. The phenyl group itself may also be substituted with 1 or more substituents, such as halogen, especially fluoro and chloro; and alkoxy, such as methoxy.

The term “alkylsulfonyl”, as used herein, means a group —SO₂R, wherein R is alkyl, especially C₁₋₇alkyl, such as methyl sulfonyl.

The term “susceptibility” refers to the sensitivity of a Gram-positive or Gram-negative bacteria for the presence of a PDF inhibitor, disclosed herein. To “increase the susceptibility” of a bacteria to the PDF inhibitor means that the bacteria will be inhibited by a lower concentration of the PDF inhibitor in the medium surrounding the bacteria.

The term “efflux pump”, as used herein, refers to a pump made of 1 or more protein components located in the cell membrane of bacteria which transports substrate molecules, e.g., antibiotics, out of the bacteria. Some efflux pumps in Gram-negative bacteria, as well as all Gram-positive multi-drug efflux pumps export substrates across a singe cytoplasmic membrane layer and are composed of 1 component, a transporter protein located in the cytoplasmic membrane. In Gram-negative bacteria, the majority of multi-drug efflux pumps contain 3 components: a transporter protein located in the cytoplasmic membrane (inner membrane), an outer membrane channel and a periplasmic linker protein connecting the two. In this arrangement, the efflux pump traverses the inner and outer membranes and thus exports substrate from the cytoplasm or cytoplasmic membrane into the external medium. The transporter proteins of the 3-component efflux pumps use proton-motive force to efflux substrates in exchange for protons, and belong to the major facilitator (MF) superfamily or to the resistance-nodulation-division (RND) family.

The term “RND efflux pump”, as used herein, refers to an efflux pump possessing a transporter protein belonging to the RND family. The predicted structure of the RND transporter protein contains 12 transmembrane helices, but also contains 2 large periplasmic domains between the transmembrane helices 1 and 2, and 7 and 8. The RND efflux pumps are able to pump out a wide range of substrates, including almost all lipophilic and amphiphilic antibiotics; chemotherapeutic agents; metabolic inhibitors, such as cerulenin; dyes; detergents, such as SDS, Triton X-100 and bile salts; and solvents. Homologous RND efflux pumps are found, e.g., In E. coli, P. aeruginosa, H. influenzae and Neisseria gonorrhoeae.

The term “efflux pump inhibitor”, as used herein, refers to a compound which specifically interferes with the ability of an efflux pump to transport substrates, e.g., antibiotics.

The term “treating”, as used herein, includes, but is not limited to, preventing, reducing and/or eliminating the clinical symptoms caused by an infection of an animal by a bacteria; preventing, reducing, and/or eliminating an infection of an animal by bacteria; or preventing, reducing, and/or eliminating contamination of an animal by bacteria. The bacteria involved is preferably Gram-negative bacteria and the animal is preferably a mammal.

The term “mammal”, as used herein, includes humans and other primates, mice, rats, sheep, cattle, horses, dogs, pigs, cats, dogs and other species.

The term “effective amount”, as used herein, means the individual amounts of a PDF inhibitor and an efflux pump inhibitor, which when utilized in combination, to treat a bacterial infection in an animal or to improve the susceptibility of bacteria to the PDF inhibitor, is sufficient to inhibit PDF. The effective amount will vary depending on the particular PDF inhibitor and efflux pump inhibitor used, the particular bacteria involved, the age, weight, sex, and medical condition of the animal, the type and severity of the infection and the route of administration, but may nevertheless be readily determined by one skilled in the art. The effective amount of a PDF inhibitor needed to be completely efficacious in treating a bacterial infection or to increase the susceptibility of bacteria is much lower than would be needed if the PDF inhibitor was administered without the efflux pump inhibitor.

The term “pharmaceutically acceptable carrier”, as used herein, means an excipient that is useful in preparing a pharmaceutical composition that is generally safe, non-toxic and neither biologically nor otherwise undesirable, and includes an excipient that is acceptable for veterinary use and human use. A “pharmaceutically acceptable carrier”, as used in the specification and claims, includes both 1 or more than 1 such carriers.

The present invention generally relates to PDF inhibitors and the finding that in bacteria, particularly Gram-negative bacteria, such as H. influenzae, E. coli, Bacillus subtilus, Klebsiella pneumoniae, P. aeruginosa and Neisseria gonorrhoeae, inactivation of genes coding for components of an RND efflux pump significantly increases the susceptibility of the bacteria to PDF inhibitors. It has also been observed that inactivation of the gene encoding a repressor protein that inhibits expression of an RND efflux pump in Gram-negative bacteria such as H. influenza, decreases susceptibility of the bacteria to the disclosed PDF inhibitors. These findings combined with other evidence below (see Examples) indicate that efflux pumps, particularly RND efflux pumps, play a major role in creating the intrinsic resistance of bacteria, particularly Gram-negative bacteria, to the PDF inhibitors. These findings further indicate that inhibition of an efflux pump in bacteria can improve the susceptibility of bacteria, particularly Gram-negative bacteria, to the PDF inhibitors.

To this end, the present invention is directed to the use of a PDF inhibitor as described below in combination with an efflux pump inhibitor, to treat bacterial infections in animals, particularly humans and other mammals and to increase the susceptibility of bacteria to a PDF inhibitor. The present invention is also directed to pharmaceutical compositions comprising the combination of a PDF inhibitor and an efflux pump inhibitor.

The term “PDF inhibitors” as used herein refer to compounds of the formula (I)

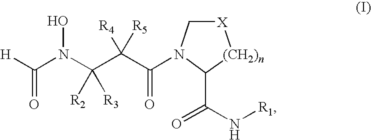

wherein
X is —CH₂—, —S—, —CH(OH)—, —CH(OR)—, —CH(SH)—, —CH(SR)—, —CF₂—, —C═N(OR)— or —CH(F)—, wherein R is alkyl;
R₁ is aryl or heteroaryl;
each of R₂, R₃, R₄ and R₅, independently, is hydrogen or alkyl, or
R₂ or R₃ and R₄ or R₅ collectively form a C₄₋₇cycloalkyl; and
n is 0-3, provided that when n is 0, X is —CH₂—,
   or a salt thereof or a prodrug thereof.

In one embodiment, R₁ is a heteroaryl of formula (II)

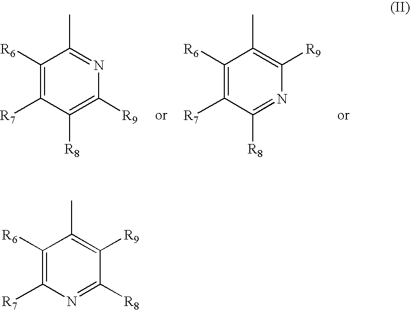

wherein each of R₆, R₇, R₈ and R₉, independently, is hydrogen, alkyl, substituted alkyl, hydroxy, alkoxy, acyl, acyloxy, SCN, halogen, cyano, nitro, thioalkoxy, phenyl, heteroalkylaryl, alkylsulfonyl or formyl.

In another embodiment, R₁ is preferably a heteroaryl of formula (II.1)

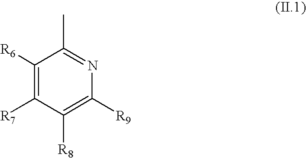

wherein R₆, R₇, R₈ and R₉ are as defined above for formula (II), e.g.,
wherein
a) R₆ is nitro, alkyl, substituted alkyl, phenyl, hydroxy, formyl, heteroalkylaryl, alkoxy, acyl or acyloxy, preferably alkyl, especially C₁₋₇alkyl, hydroxyl or alkoxy, especially a C₁₋₇alkoxy; and
   R₇, R₈ and R₉ are hydrogen, or
b) R₆, R₈ and R₉ are hydrogen; and
   R₇ is alkyl, substituted alkyl, phenyl, halogen, alkoxy or cyano (preferably alkyl, especially C₁₋₇alkyl), substituted alkyl (especially substituted C₁₋₇alkyl, such as —CF₃ or alkoxy (especially C₁₋₇alkoxy), or
c) R₆, R₇ and R₉ are hydrogen; and
   R₈ is alkyl, substituted alkyl, halogen, nitro, cyano, thioalkoxy, acyloxy, phenyl, alkylsulfonyl or carboxyalkyl (preferably alkyl, especially C₁₋₇alkyl), substituted alkyl (especially —CF₃), halogen or carboxyalkyl, or
d) R₆, R₇ and R₈ are hydrogen; and
   R₉ is alkyl, halogen or hydroxy, or
e) R₇ and R₉ are hydrogen; and
   each of R₆ and R₈, independently, is halogen, alkyl, substituted alkyl, phenyl or cyano, or
f) each of R₇ and R₈ is alkyl or substituted alkyl; and
   R₆ and R₈ are hydrogen, or
g) R₆ and R₈ are hydrogen;
   R₇ is alkyl or substituted alkyl; and
   R₈ is nitro, or
h) R₈ and R₉ are hydrogen;
   R₆ is cyano; and
   R₇ is alkoxy, or
i) R₇ and R₈ are hydrogen; and
   R₆ is alkyl, substituted alkyl, alkoxy or SCN; and
   R₉ is alkyl or substituted alkyl, or
j) R₆ and R₇ are hydrogen;
   R₈ is nitro or halogen; and
   R₉ is alkyl or substituted alkyl, or
k) R₆, R₇, R₈ and R₉ are hydrogen, or
l) R₆ and R₇, together with the carbon atoms to which they are attached, form a phenyl group, preferably substituted with hydroxy; and
   R₈ and R₉ are hydrogen, or
m) R₆ and R₇ are hydrogen; and
   R₈ and R₉, together with the carbon atoms to which they are attached, form a phenyl group, or
n) n is 0, or
o) n is 0; and
   each of R₆, R₇, R₈ and R₉, independently, is hydrogen, alkyl or halogen and, more particularly R₆, R₇, R₈ and R₉ are hydrogen, or
p) n is 0;
   R₆, R₈ and R₉ are hydrogen; and
   R₇ is alkyl, or
q) n is 0;
   R₆R₇ and R₉ are hydrogen; and
   R₈ is alkyl or halogen.

In another embodiment, R₁ is of formula (II.2)

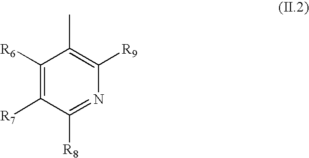

wherein
R₆, R₇, R₈ and R₉ are as defined above for formula (II), in particular, R₇ and R₈, together with the carbon atoms to which they are attached, form a phenyl group; and
R₆ and R₉ are hydrogen.

In yet another embodiment, the R₁ is of formula (III)

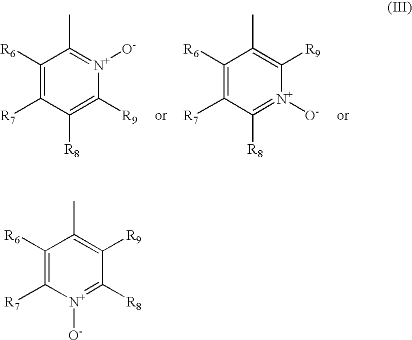

wherein
each of R₆, R₇, R₈ and R₉, independently, is hydrogen, alkyl, substituted alkyl, phenyl, halogen, hydroxy or alkoxy, e.g.,
wherein
a) R₆ and R₈ are hydrogen;
   R₉ is hydrogen or alkyl; and
   R₇ is alkyl, substituted alkyl or phenyl, or
b) R₆, R₇ and R₉ are hydrogen; and
   R₈ is halogen, alkyl or substituted alkyl, or
c) R₇, R₈ and R₉ are hydrogen; and
   R₆ is hydroxy.

In a particularly useful embodiment, the heteroaryl is of formula (III.1)

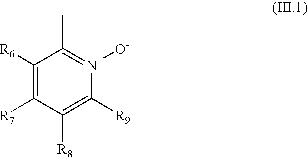

wherein R₆, R₇, R₈ and R₉ are as defined above for formula (III).

In another embodiment, R₁ is an unsubstituted phenyl or the phenyl is substituted with alkoxy, e.g., methoxy; or aryloxy, e.g., phenoxy.

In a particularly useful embodiment, the compound of formula (III.1) is

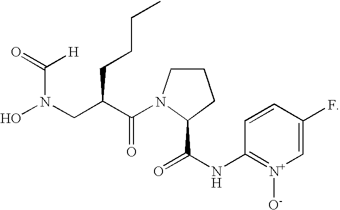

In another embodiment, the R₁ is of formula (IV)

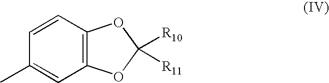

wherein each of R₁₀ and R₁₁, independently, is hydrogen or halogen, in particular, R₁₀ and R₁₁, are both either hydrogen or both halogen.

It will be appreciated that the compounds of formula (I) may exist in the form of optical isomers, racemates or diastereoisomers. For example, a compound of formula (I), wherein R₂ and R₃ are different residues, or wherein R₄ and R₅ are different residues, is asymmetric and may have the R- or S-configuration. It is to be understood that the compounds of formula (I) embrace all enantiomers and their mixtures. Similar considerations apply in relation to starting materials exhibiting assymetric carbon atoms as mentioned.

The compounds of formula (I), may exist in free form or in salt form, e.g., in form of a pharmaceutically acceptable salt. A “pharmaceutically acceptable salt” of a compound means a physiologically and pharmaceutically acceptable salt that possesses the desired pharmacological activity of the parent compound and does not impart undesired toxicological effects. Such salts include:
(1) acid addition salts formed with inorganic acids, such as hydrochloric acid, hydrobromic acid, sulfuric acid, nitric acid, phosphoric acid and the like; or formed with organic acids, such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-napthalenesulfonic acid, 4-toluenesulfonic acid, camphorsulfonic acid, 3-phenylpropionic acid, trimethylacetic acid, tertiary butylacetic acid, lauryl sulfuric acid, gluconic acid, glutamic acid, hydroxynapthoic acid, salicylic acid, stearic acid, muconic acid and the like; or
(2) salts formed when an acidic proton present in the parent compound either is replaced by a metal ion, e.g., an alkali metal ion, an alkaline earth ion or an aluminum ion; or coordinates with an organic base, such as ethanolamine, diethanolamine, triethanolamine, tromethamine, N-methylglucamine and the like.

The compounds of formula (I) may act as a pro-drug. “Prodrug” means any compound which releases an active parent drug according to formula (I) in vivo when such prodrug is administered to a mammalian subject. Prodrugs of a compound of formula (I) are prepared by modifying functional groups present in the compound of formula (I) in such a way that the modifications may be cleaved in vivo to release the parent compound. Prodrugs include compounds of formula (I), wherein a hydroxy, amino or sulfhydryl group is bonded to any group that may be cleaved in vivo to regenerate the free hydroxyl, amino or sulfhydryl group, respectively. Examples of prodrugs include, but are not limited to, esters, e.g., acetate, formate and benzoate derivatives; carbamates, e.g., N,N-dimethylamino-carbonyl; of hydroxy functional groups in compounds of formula (I) and the like.

The compounds of formula (I) can be obtained by synthetic chemistry methods know to those skilled in the art and as described, e.g., in WO 02/102790 A1.

In one aspect, the present invention provides a method for treating bacterial infections, particularly Gram-negative bacterial infections, in an animal, preferably a mammal. The method includes administering an effective amount of a PDF inhibitor, i.e., a compound embraced by the formula (I) or a salt thereof or a prodrug thereof, and an efflux pump inhibitor.

The combination of a PDF inhibitor and efflux inhibitor increases the susceptibility of the Gram-negative bacteria for the PDF inhibitor. Accordingly, in using the combination of PDF inhibitor and efflux pump inhibitor, a Gram-negative infection caused by bacteria which are resistant, i.e., have decreased susceptibility, to a particular PDF inhibitor in the absence of the efflux pump inhibitor may be treatable with that particular PDF inhibitor In addition, Gram-negative infections in which the bacteria are susceptible to the PDF inhibitors in the absence of an efflux inhibitor, can be treated utilizing lower dosages of the PDF inhibitor, thus, mitigating the risk of side effects that can occur from utilizing high dosages of the PDF inhibitor.

As stated above, the majority of the Gram-negative bacteria possess a 3-component efflux pump consisting of a transporter protein located in the cytoplasmic membrane (inner membrane), an outer membrane channel and a periplasmic linker protein connecting the two. See Nikaido (1996), supra; Nikaido, Clin Infect Dis, Vol. 27 (Supp. 1), pp. S32-S41 (1998); and Zgurskaya and Nikaido, Mol Microbiol, Vol. 37, No. 2, pp. 219-225 (2000). In this arrangement, the efflux pump traverses the inner and outer membranes and thus exports substrate such as an antibiotic from the cytoplasm or cytoplasmic membrane into the external medium. See Zgurskaya and Nikaido (2000), supra. The transporter proteins of the 3-component efflux pumps use proton-motive force to efflux substrates in exchange for protons, and belong to the MF superfamily or to the RND family. See Nikaido (1998), supra.

In one embodiment, the Gram-negative bacterial infection to be treated is caused by Gram-negative bacteria possessing an RND efflux pump. As stated above, the term “RND efflux pump” refers to an efflux pump possessing a transporter protein belonging to the RND family, which includes in addition a putative transporter of nodulation signal molecules in Rhizobium. See Saier et al., FASEB J, Vol. 12, pp. 265-274 (1998); and Saier et al., Mol Microbiol, Vol. 11, pp. 841-847 (1994). All members of the RND efflux pump family have similar structures. Their proposed structure contains 12 transmembrane helices and also contains 2 large periplasmic domains between the transmembrane helices 1 and 2, and 7 and 8. See Nikaido (1998), supra; and Zgurskaya and Nikaido (2000), supra. The RND efflux pumps are able to pump out a wide range of substrates, including almost all lipophilic and amphiphilic antibiotics; chemotherapeutic agents; metabolic inhibitors, such as cerulenin; dyes; detergents, such as SDS, Triton X-100 and bile salts; and solvents. See Nikaido, Curr Opin Microbiol (1998), supra; and Nikaido (1996), supra. Most of the efflux transporters that mediate resistance to clinically relevant antibiotics are members of RND family. See Saier et al., FASEB J (1998), supra. Gram-negative bacteria possessing an RND efflux pump include, but are not limited to, Serratia marcescens, E. coli, Moraxella catarrhalis, Bacillus subtilus, Klebsiella pneumoniae, P. aeruginosa, H. influenzae, Neisseria gonorrhoeae, Burholderia cepacia, Burkholderia pseudomallei, Stenotrophomonas maltophilia, Acinetobacter baumannii (A. baumannii) and Pseudomonas putida. Homologous efflux RND pumps can be found in Gram-negative bacteria such as E. coli, P. aeruginosa, H. influenzae and Neisseria gonorrhoeae. See Nikaido, Curr Opin Microbiol (1998), supra. In a particular useful embodiment, the Gram-negative bacteria possessing an RND pump is H. influenzae.

In particularly useful embodiments of the method for treating a Gram-negative bacterial infection, the compound of formula (I) is

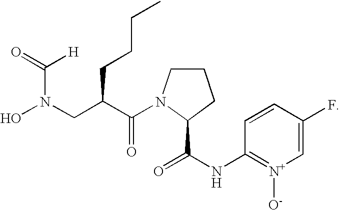

Efflux pump inhibitors specific for efflux pumps, particularly RND efflux pumps, in Gram-negative bacteria are well-known in the art. For example, U.S. Pat. No. 6,114,310 describes a generic class of efflux pump dipeptide inhibitors that enhance the activity of a wide range of antibiotics against a variety of Gram-negative bacteria. An example of a dipeptide efflux pump inhibitor within this generic class is MC-04,124 having the structure

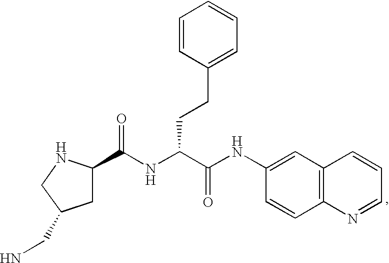

which has been shown to inhibit RND efflux pumps and enhance the activity of several antibiotics against a variety of Gram-negative bacteria, such as E. coli, Klebsiella pneumoniae, P. aeruginosa, H. influenzae and A. Baumannii. See Cho et al., 40^{th} ICAAC (2000); and Watkins et al., Bioorgan Med Chem Lett, Vol. 13, pp. 4241-4244 (2003). Other examples of RND efflux pump inhibitors that enhance the activity of antibiotics against Gram-negative bacteria include the dipeptide inhibitor, MC-207,110 having the structure

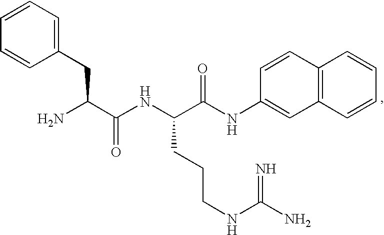

as described in Renau et al., J Med Chem, Vol. 42, pp. 4928-4931 (1999); and Lomovskaya et al., Antimicrob Agents Chem, Vol. 45, No. 1, pp. 105-116 (1991), and the dipeptide inhibitor, MC-002,595, having the structure

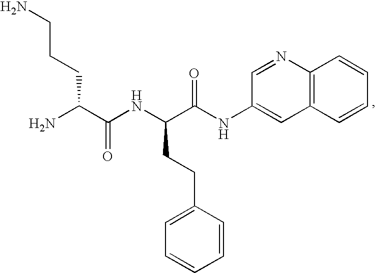

as described in Lomovskaya et al., Antimicrob Agents Chem (1991), supra.

Another example of RND efflux pump inhibitors that enhance the activity of oxazolidinone antibacterial agents against Gram-negative bacteria are arginine derivatives as described in U.S. Pat. No. 6,251,869.

Yet another example of efflux pump inhibitors specific for RND efflux pump in Gram-negative bacteria, such as P. aeruginosa are sulfates of benastatins A and B produced by an actinomycete, e.g., MF-EA-371α having the structure

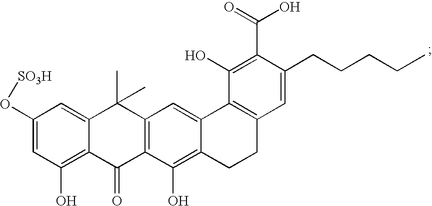

and MF-EA-371δ having the structure

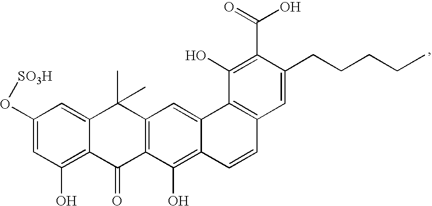

as described in Lee et al. (2000), 40^{th} ICAAC (2000).

For treatment of a Gram-negative bacterial infection in humans and other animals, particularly humans and other mammals. that have been diagnosed as having a Gram-negative bacterial infection, a PDF inhibitor and an efflux pump inhibitor or pharmaceutical compositions thereof, can be administered by any conventional route, e.g., locally or systemically, e.g., orally, topically, parenterally, subdermally or by inhalation. The route chosen will depend on the type, severity and location of the infection and the type of bacteria causing the infection.

The required dosage of PDF inhibitor to achieve an “effective amount” will of course depend on various factors as described above, e.g., the mode of administration, the type and severity of infection to be treated and the effect desired. Generally, an effective amount of dosage, e.g., for human treatment, will range, for example, from about 1-3000 mg per day, for instance 1500 mg per day depending on the route and frequency of administration. Such a dosage corresponds to about 0.015-50 mg/kg of body weight per day. Suitably the dosage, is for example, from about 5-20 mg/kg of body weight per day.

The amount of a particular efflux pump inhibitor to be used will depend on various factors, e.g., the type of Gram-negative bacteria, susceptibility of the Gram-negative bacteria to the particular PDF inhibitor, and the absorption by the animal being treated. Sufficient amounts of the efflux pump inhibitor should be used to make the Gram-negative bacteria susceptible to a pharmaceutically acceptable level of the PDF inhibitor in the treated animal. The sufficient amount of a particular efflux inhibitor can be readily determined by testing for minimum inhibitory concentration (MIC) of the PDF inhibitor and comparing the MIC of that PDF inhibitor alone, with the MIC of that PDF inhibitor utilized in combination with the efflux pump inhibitor. Generally, the molar ratio of an efflux pump inhibitor to a PDF inhibitor which is administered is from about 0.01 to 10. Suitably the molar ratio is from about 0.1 to 1.0. Accordingly, the daily dosage of an efflux pump inhibitor for treating a gram-negative bacterial infection in an animal can range from about 0.0015-5 mg/kg of body weight per day. Suitably, the daily dosage is from about 0.5-2 mg/kg of body weight per day. The efflux pump inhibitor can be administered before the PDF inhibitor is administered, or can be administered simultaneously with the PDF inhibitor.

In another aspect, the present invention provides a method for increasing the susceptibility of Gram-negative bacteria to a PDF inhibitor, comprising contacting the bacteria with a PDF inhibitor and an efflux pump inhibitor, in an amount effective to Inhibit an efflux pump in the Gram-negative bacteria. The method improves the effectiveness of the PDF inhibitor against the Gram-negative bacterial cell which expresses an efflux pump when treated with the combination of a PDF inhibitor and efflux pump inhibitor.

In one embodiment of the method for increasing the susceptibility of a Gram-negative bacteria to a PDF inhibitor, the Gram-negative bacterial infection is caused by a Gram-negative bacteria possessing an RND efflux pump, e.g., Serratia marcescens, E. coli, Moraxella catarrhalis, Bacillus subtilus, Klebsiella pneumoniae, P. aeruginosa, H. influenzae, Neisseria gonorrhoeae, Burholderia cepacia, Burkholderia pseudomallei, Stenotrophomonas maltophilia, Acinetobacter baumannii and Pseudomonas putida. In a further embodiment, the Gram-negative bacteria possessing an RND efflux pump includes E. coli, P. aeruginosa, H. influenzae and Neisseria gonorrhoeae. In a particular useful embodiment, the Gram-negative bacteria possessing an RND efflux pump is H. influenzae. Particularly useful PDF inhibitors and efflux pump inhibitors that can be utilized in this method are as described above.

Efficacy of a particular efflux pump inhibitor in increasing the susceptibility of Gram-negative bacteria to a particular PDF inhibitor can be evaluated e.g., in vitro using the checkerboard method as described in Lorian, ed., Antibiotics in Laboratory Medicine, 3^{rd} Ed., p. 432, Williams & Wilkins, Baltimore, Md.

In a further aspect, the present invention provides pharmaceutical compositions effective for treatment of a bacterial infection, particularly a Gram-negative bacterial infection, in an animal, e.g., a mammal. The composition includes a PDF inhibitor and efflux pump inhibitor as described above and a pharmaceutically acceptable carrier.

The compositions may be in any form known in the art including, but not limited to, tablets, capsules, wafers, fast melts (without wafers), powders, granules, lozenges, creams or liquid preparations, such as oral or sterile parenteral solutions or suspensions. The PDF inhibitor and efflux pump inhibitor may also be administered in liposomal, micellar or microemulsion formulations. The PDF inhibitor present in the composition may also be administered as prodrugs, where the prodrug administered undergoes biotransformation in the treated mammal to a form which is biologically active.

The topical formulations of the present invention may be presented as, i.e., ointments; creams or lotions; solutions; salves; emulsions; plasters; eye ointments and eye or ear drops; impregnated dressings; transdermal patches; sprays and aerosols; and may contain appropriate conventional additives, such as preservatives; solvents to assist drug penetration; and emollients in ointments and creams.

The formulations may also contain compatible conventional carriers, such as cream or ointment bases and ethanol; or oleyl alcohol for lotions. Such carriers may be present, e.g., from about 1% up to about 99% of the formulation. For example, they may form up to about 80% of the formulation.

Tablets and capsules for oral administration may be in unit dose presentation form, and may contain conventional excipients, such as binding agents, e.g., syrup, acacia, gelatin, sorbitol, tragacanth or polyvinylpyrollidone; fillers, e.g., lactose, sugar, maize-starch, calcium phosphate, sorbitol or glycine; tabletting lubricants, e.g., magnesium stearate, talc, polyethylene glycol or silica; disintegrants, e.g., potato starch; or acceptable wetting agents, such as sodium lauryl sulphate. The tablets may be coated according to methods well-known in standard pharmaceutical practice.

Oral liquid preparations may be in the form of, for example, aqueous or oily suspensions, solutions, emulsions, syrups or elixirs, or may be presented as a dry product for reconstitution with water or another suitable vehicle before use. Such liquid preparations may contain conventional additives, such as suspending agents, e.g., sorbitol, methyl cellulose, glucose syrup, gelatin, hydroxyethyl cellulose, carboxymethyl cellulose, aluminium stearate gel or hydrogenated edible fats; emulsifying agents, e.g., lecithin, sorbitan monooleate or acacia; non-aqueous vehicles (which may include edible oils), e.g., almond oil; oily esters, such as glycerine, propylene glycol or ethyl alcohol; preservatives, e.g., methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The PDF inhibitor and efflux pump inhibitor, depending on the vehicle and concentration used, may be either suspended or dissolved in the vehicle or other suitable solvent. In preparing solutions, the PDF and efflux inhibitors may be dissolved in water for injection and filter sterilized before filling into a suitable vial or ampule and sealing. Advantageously, agents, such as a local anesthetic preservative and buffering agents, may be dissolved in the vehicle. To enhance the stability, the composition may be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the PDF inhibitor and efflux pump inhibitor are suspended in the vehicle instead of being dissolved and sterilization cannot be accomplished by filtration. The PDF and efflux inhibitor may be sterilized by exposure to ethylene oxide before suspending in the sterile vehicle. Advantageously, a surfactant or wetting agent is included in the composition to facilitate uniform distribution of the compound.

The PDF inhibitor may be administered in free form or in pharmaceutically acceptable salt form, e.g., as indicated above. Such salts may be prepared in conventional manner and exhibit the same order of activity as the free compounds.

The following examples serve to illustrate the invention but do not to limit the scope thereof in any way.

### EXAMPLES

### Methods and Materials

### Bacterial Strains, Plasmids and Growth Media

The bacterial strains and plasmids used in this study are listed in Table 1 below. L-broth or L-agar (Difco) is used for routine growth of Escherichia coli (E. coli). Chocolate agar plates (Remel) are used for routine growth of H. influenzae. Brain heart infusion broth (Remel) supplemented with 10 μg/mL of β-NAD (Fluka) and 10 μg/mL hemin/vitamin K solution (sBHI) (Remel) is used for liquid broth cultivation of H. influenzae. For induction of natural competence in H. influenzae, nutritional downshift is induced using M-IV medium as described previously. See Poje and Redfield, Methods Mol Med, Vol. 71, No., pp. 57-70 (2003). Kanamycin is added to growth media at 50 μg/mL (E. coli) or 5 μg/mL (H. influenzae) as required.

TABLE 1
Bacterial Strains and Plasmids Used in This Study

| | | Reference or |
|---|---|---|
| Strain or Plasmid | Genotype or Relevant Characteristics | Source |
| H. influenzae | | |
| NB65044 | Rd KW20 | Fleischmann* |
| NB65001 | | ATCC 49247 |
| NB65062 | Clinical isolate, LBM415 hyper-susceptible | Jones |
| NB65016 | Clinical isolate, decreased LBM415 susceptibility | Chopra |
| NB65027 | Clinical isolate, decreased LBM415 susceptibility | Willinger |
| NB65051 | Clinical isolate, decreased LBM415 susceptibility | Jones |
| NB65063 | Clinical isolate, decreased LBM415 susceptibility | Jones |
| NB65069 | Clinical isolate, decreased LBM415 susceptibility | Jones |
| NB65076 | Clinical isolate, decreased LBM415 susceptibility | Jones |
| NB65062-CDS0037 | NB65062 derivative, complemented for acrA deficiency | This study |
| NB65062-CDS0038 | NB65062 derivative, complemented for acrA deficiency | This study |
| NB65044-CDS0001 | NB65044 acrB::Km | This study |
| NB65044-CDS0020 | NB65044 acrB::Km | This study |
| NB65044-CDS0008 | NB65044 acrR::Km | This study |
| NB65044-CDS0011 | NB65044, acrR mutant selected on 8 μg/mL LBM415 | This study |
| NB65044-CDS0014 | NB65044, acrR mutant selected on 8 μg/mL LBM415 | This study |
| NB65016-CDS0004 | NB65016 acrB::Km | This study |
| NB65027-CDS0021 | NB65027 acrB::Km | This study |
| NB65027-CDS0003 | NB65027 HI1462::Km | This study |
| NB65051-CDS0002 | NB65051 acrB::Km | This study |
| NB65051-CDS0022 | NB65051 acrB::Km | This study |
| NB65063-CDS0005 | NB65063 acrB::Km | This study |
| NB65069-CDS0007 | NB65069 acrB::Km | This study |
| NB65076-CDS0006 | NB65076 acrB::Km | This study |
| E. coli | | |
| Top 10 | | Invitrogen |
| Plasmids | | |
| pCR 2.1 topo | Cloning vector | Invitrogen |
| pEX18Tc | Cloning vector | Hoang** |
| pCDBKmUS | pEX18Tc derivative containing the H. influenzae uptake | This study |
| | sequence and acrB interrupted by a TN903 derived Km^{r} | |
| | cassette | |
| pCD14Km | pBluescript containing ORF HI1462 interrupted by a | This study |
| | TN903 derived Km^{r} cassette | |
| pCDRKm | pBluescript containing acrR interrupted by a TN903 | This study |
| | derived Km^{r} cassette | |
| pACYC177 | Cloning vector, source of TN903 Km^{r} cassette | NEB |
| pACYC184 | Cloning vector, replicates in H. influenzae | NEB |
| pBAD18 | Cloning/expression vector, source of TN903 Km^{r} marker | Beckwith |
| pBluescript SK | Cloning vector | |

*See Fleischmann et al. (1995), supra.
**See Hoang, Karkhoff-Schweizer, Kutchma and Schweizer, Gene, Vol. 212, No. 1, pp. 77-86 (1998).

### Antimicrobial Susceptibility Testing

Antibiotic and substrate MICs are determined by broth microdilution using 2-fold dilution in HTM in accordance with the procedures established by the National Committee for Clinical Laboratory Standards (NCCLS). See NCCLS, Approved standard M7-A5, Wayne, Pa. (2003). PDF inhibitors are synthesized at the Novartis Institutes for BioMedical Research, Cambridge, Mass. All remaining antibiotics are obtained from Sigma (St. Louis, Mo.).

### DNA Manipulations

H. influenzae genomic DNA is isolated using the Puregene tissue kit from Gentra Systems Inc. (Minneapolis, Minn.) according to the instructions. Oligonucleotides for PCR and sequencing are obtained from Genelink (Hawthorne, N.Y.), and PCR reactions are carried out using the Easystart mix-in-a-tube system from Molecular Bio-Products Inc. (San Diego, Calif.) according to the supplied instructions. Prepared genomic DNA or cells from isolated colonies are used as template in PCR reactions. Restriction endonucleases and modifying enzymes are used according to the instructions supplied with the enzymes. DNA fragments are purified, or isolated following agarose gel electrophoresis, using the QIAquick PCR cleanup or gel extraction kits from Qiagen Inc. (Valencia, Calif.) as specified in the instructions. Nucleotide sequencing is performed by Agencourt Inc. (Waltham, Mass.).

### In Vitro Mutagenesis and Gene Replacement

To construct an insertion knockout of the acrB homolog in H. influenzae, primers acrBHIF (5′-CCACTTTAATGTATGAGGAAATCCG-3′) (SEQ ID NO. 1) and acrBHIR3 (5′-CGAATTACGTAAGATAACCTAAGTGCG-3′) (SEQ ID NO. 2) are used to generate a 3.1 kb PCR fragment encompassing the acrB gene using H. influenzae NB65001 genomic template DNA. The acrB PCR product is ligated into PCR 2.1-Topo by Invitrogen (Carlsbad, Calif.), according to the instructions provided with the kit, then the acrB fragment is excised using EcoRI and ligated into pEX18Tc. This construct is then linearized at the unique Mfel site within the acrB fragment, blunt-ended with T4 DNA polymerase and ligated to a 1.2 kb blunt PCR fragment encompassing the TN903 kanamycin resistance determinant generated from pACYC177 using primers TN903KanF (5′-GCCACGTTGTGTCTCAAAATCTCTG-3′) (SEQ ID NO. 3) and TN903KanR (5′CCAGTGTTACACCAATTAACCAA-3′) (SEQ ID NO. 4), to give plasmid pCDBKm. A 177 bp DNA fragment containing the H. influenzae DNA uptake sequence is PCR amplified from NB65044 genomic DNA using previously described primers. See Akerley et al., Proc Natl Acad Sci USA, Vol. 99, No. 2, pp. 966-971 (2002). The product is cloned into PCR2.1-Topo by Invitrogen, according to the instructions, then excised with KpnI and ligated into the KpnI site within the multi-cloning site of pCDBKm to give pCDBKmUS. Inclusion of the uptake sequence has been shown to facilitate much greater levels of transformation, which makes it useful for introducing DNA into various isolates which may not efficiently take up linear DNA by natural transformation. See Akerley et al. (2002), supra.

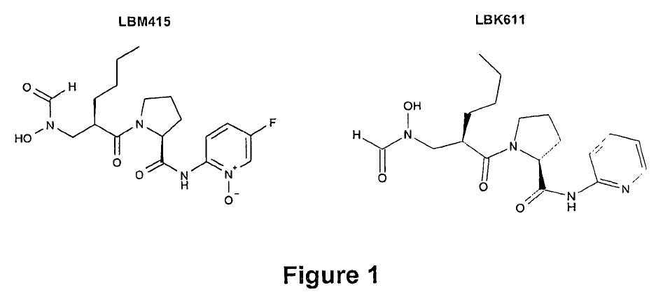

To construct an insertion knockout of open-reading frame (ORF) HI1462 (toIC) [see Trepod and Mott, Antimicrob Agents Chemother, Vol. 48, No. 4, pp. 1416-1418 (2004)], primers HI1462IF (5′-CACGCTTGCTTTGTTGATGTCTGGTGC-3′) (SEQ ID NO. 5) and HI1462IR (5′-TCCCGCCATTGAGCTATATACCGCA-3′) (SEQ ID NO. 6) are used to generate a 1.3 kb PCR fragment encompassing most of HI1462 from NB65001 genomic DNA template and ligated directly into PCR 2.1-Topo, by Invitrogen, according to the instructions. The insert is then recovered as an EcoRI fragment and ligated into the EcoRI site of pBluescriptSK. A TN903 kanamycin resistance determinant, isolated from pBAD18Tc as a 1.8 kb Haell fragment and blunt-ended with T4 DNA polymerase, is then ligated into the unique MluI site within HI1462, which had been rendered blunt, to generate pCD14 Km. This construct has the kanamycin resistance determinant in the same orientation as HI1462. See FIG. 1.

To construct an insertion knockout of ORF H10893, an acrR homolog and putative repressor of acrAB expression, primers acrRHIF2 (5′-TAATGATGAAAAGTGCGGTTAATT-3′) (SEQ ID NO. 7) and acrRHIR (5′-TTTCTGAATCGCACGCCAAGAGCGT) (SEQ ID NO. 8) are used to generate a 752 bp PCR fragment encompassing ORF HI0893 from NB65001 genomic DNA. This PCR fragment is ligated into PCR 2.1 Topo, by Invitrogen, according to the instructions provided with the kit, then excised as an EcoRI fragment and cloned into EcoRI cut pBluescriptSK. The construct is then linearized with Sful and ligated to a 1.2 kb blunt PCR fragment encompassing the TN903 kanamycin resistance determinant, generated from pACYC177 using the primers described above, to give plasmid pCDRKm.

To introduce the acrB::Km and acrR::Km insertions into the genome, H. influenzae strains is grown to early log phase (OD₆₀₀ approximately 0.2) in sBHI and natural competence is induced by nutritional downshift into M-IV medium as described by Poje and Redfield (2003), supra. Competent cells are transformed with pCDBKmUS (linearized with XbaI) or pCDRKm (linearized with ScaI), as previously described [see Poje and Redfield (2003), supra], and plated on chocolate agar containing 5 μg/mL kanamycin to select for cells containing the kanamycin resistance marker on their genome. To introduce the HI1462::Km insertion into the genome of strain NB65044, competent cells are transformed with pCD14Km (linearized with ScaI) and selected on chocolate agar plates, as described above. To introduce this insertion into strains NB65027 and NB65051, the recipient cells are made competent as described above, transformed with genomic DNA isolated from strain NB65044-CDS0001 and selected on chocolate agar plates containing 5 μg/mL kanamycin. Insertions are confirmed by PCR and sequencing of fragments generated using primers flanking the insertion site.

PCR Analysis of Hyper-Susceptible Strain NB65062 and Complementation of the acrA Deletion

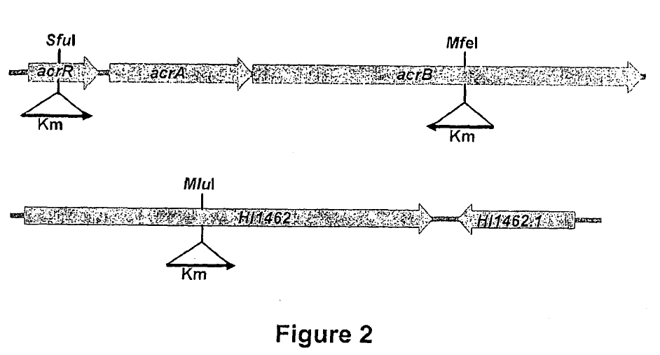

To obtain the region encompassing the acrA gene in strain NB65062, primers acrRHIF1 (5′-GTGCGGTGCCACCGCAAGGACATA-3′) (SEQ ID NO. 9) and acrAHIR (5′-TGCAGGCTCTATTGCACCCACAATG-3′) (SEQ ID NO. 10) are used to generate a PCR fragment from NB65062 genomic DNA. See FIG. 2. The fragment is gel purified and the nucleotide sequence is determined.

For complementation of the acrA defect, genomic DNA from NB65044, which contains a functional acrAB pump, is used to transform NB65062 using the nutritional downshift method described above. Transformed cells are plated on chocolate agar containing either 4 μg/mL LBM415 or 2 μg/mL erythromycin, both substrates of the acrAB efflux pump.

### Example 1

### Role of the acrAB Pump in Decreasing Intrinsic Susceptibility to LBM415

Insertional inactivation of acrB in the laboratory strain NB65044 (see FIG. 1), significantly increases susceptibility to LBM415, reflects in the MIC of LBM415 dropping from 4 μg/mL against the parent strain to 0.25 μg/mL against the acrB-deficient derivative NB65044-CDS0001. See Table 2 below. The insertion also increases susceptibility to LBK611 and the known pump substrate erythromycin [see Sanchez, Pan, Vinas and Nikaido (1997), supra), and more dramatically to another macrolide (clindamycin), while not significantly affecting susceptibility to the pump non-substrate [see Sanchez, Pan, Vinas and Nikaido (1997), supra], tetracycline. See Table 2. Another known pump non-substrate, chloramphenicol, is also unaffected by loss of acrB (data not shown). This confirms that the AcrAB pump of H. influenzae is a major driver of reduced susceptibility to LBM415.

TABLE 2
Contribution of the AcrAB Efflux Pump to Intrinsic Resistance
to LBM415, LBK611 and Macrolides in H. influenzae

| | MIC (μg/mL) | | | | |
|---|---|---|---|---|---|
| Strain | LBM415 | LBK611 | ERM | CLD | TET |
| NB65044 | 4 | 4 | 4 | 8 | 0.5 |
| NB65044-CDS0001 | 0.25 | 0.25 | 0.25 | 0.5 | 0.5 |
| NB65044-CDS0020 | 0.125 | 0.125 | 0.25 | 0.5 | 1 |
| NB65062 | ≦0.25 | ND | ND | ≦0.25 | 0.5 |
| NB65062-CDS0037 | 8 | ND | ND | 16 | 1 |
| NB65062-CDS0038 | 8 | ND | ND | 16 | 1 |

ERM = erythromycin
CLD = clindamycin
TET = tetracycline
ND = not done

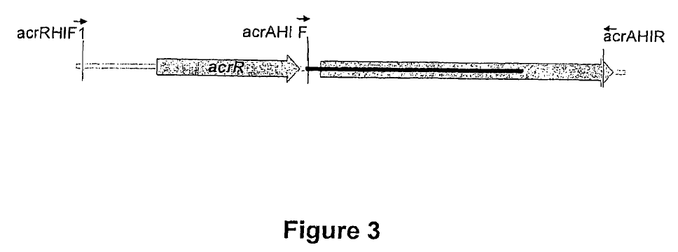

Efflux has previously been implicated in mediating moderate levels of macrolide resistance in H. influenzae clinical isolates based on a relative lack of accumulation of radiolabelled erythromycin in resistant strains compared to susceptible strains, whereas high level resistance to macrolides is associated with target mutations. See Peric, Bozdogan, Jacobs and Appelbaum (2003), supra. A small proportion of clinical strains previously examined [see Peric, Bozdogan, Jacobs and Appelbaum (2003), supra] are hyper-susceptibility to macrolides and do not accumulate radiolabelled erythromycin, suggesting that they lack an efflux pump. It is also noticed that a small percentage of isolates are hyper-susceptible to both macrolides and PDF inhibitors. Using PCR diagnostics, acrR-, acrA- or acrB-derived fragments are obtained for several of these strains (data not shown), suggesting that the pump genes are widely-distributed, even in hyper-susceptible strains. For hyper-susceptible in strain NB65062 however (see Table 2), no acrA product is obtained, indicating that there might be a genomic deletion. Generation of a PCR fragment encompassing the acrA region using flanking primers (see FIG. 3) resulting in a fragment of substantially smaller size than the predicted 2 kb. Nucleotide sequencing of the fragment reveals an 873 bp deletion (see FIG. 3) resulting in the loss of most of acrA. Transformation of NB65062 with genomic DNA from NB65044, which possesses an intact acrAB locus, and selection on chocolate agar containing LBM415 (4 μg/mL) or erythromycin (2 μg/mL), results in isolates with decreased susceptibility to both classes of antibiotic and no change in susceptibility to pump non-substrates strains NB65062-CDS0038 and NB65044-CDS0039. See Table 2. Pulse field gel electrophoresis analysis of the transformants give identical restriction patterns to that of NB65062, while PCR and sequencing reveal the restoration of full-length acrA, confirming that hyper-susceptibility is indeed the result of a lack of the acrAB pump in strain NB65062. This is consistent with the role of the AcrAB pump in providing intrinsic resistance to PDF inhibitors and macrolides, and implies that rather than failing to acquire the efflux pump, as suggested previously [see Peric, Bozdogan, Jacobs and Appelbaum (2003), supra], macrolide and LBM415 hyper-susceptible strains may result from mutational loss of efflux pump components. Sequencing of the acrR genes from these strains also reveal no difference from the parent strain indicating that an acrR mutation is not also selected during exposure to either LBM415 or erythromycin in the selective plates. The remaining hyper-susceptible strains that give predicted PCR products for various acr genes may have other less obvious mutations compromising pump function, but this remains to be confirmed.

Given the identification of only one RND family pump in H. influenzae to date, it might be expected that it serves an important function in survival in the human host, the only natural environment known for H. influenzae. The loss of this pump in certain clinical isolates is curious in that the pump is generally maintained in H. influenzae but apparently can be lost in certain instances, suggesting its role is not indispensable.

### Example 2

### Identification of the Outer Membrane Component of the acrAB Homolog Efflux Pump

Having confirmed that the acrAB pump is a major contributor to intrinsic resistance to both PDF inhibitors and macrolides in H. influenzae, it was of interest to identify the outer membrane channel component of the pump to complete the pumps tripartite architecture. Protein homology searches of the H. influenzae genome using the ToIC outer membrane channel that partners with acrAB in E. coli, reveals significant similarity to ORF HI1462 (expectation value 2.9×10⁻⁶). Interestingly, the oprM component of the P. aeruginosa mexAB-oprM pump yields an even closer match to HI1462 with an expectation value of 2.8×10⁻²², indicating that HI1462 is a good candidate for the outer membrane channel. Consistent with this, inactivation of HI1462 (see FIG. 1) in H. influenzae NB65044 increases susceptibility to erythromycin, clindamycin and LBM415, all substrates of the acrAB pump, while not affecting susceptibility to the pump non-substrate tetracycline NB65044-CDS0022. See Table 2. Recently, another group [see Trepod and Mott (2004), supra], has also reported the identification of HI1462 as the outer membrane channel for this pump. The data included here therefore supports this finding and extends the role of this channel component in mediating resistance to LBM415.

Interestingly, HI1462 shows total identity over its C-terminal encoding half to another ORF HI1340. Despite this similarity, the clear impact of losing HI1462 on specific susceptibility to pump substrates confirms its role as the channel, with perhaps little clinically relevant contribution from HI1340. The differences between HI1462 and HI1340 over their N-terminal portions may specify interactions with different pumps, at least in NB65044. Indeed, there is an emrAB pump homolog HI0898 and HI0899, respectively, separate from the acrAB pump only by one gene, ftsN, that could function with HI1340. Inactivation of the emrAB pump in H. influenzae does not affect susceptibility to a wide range of compounds [Trepod and Mott (2004), supra], suggesting that this pump might not efficiently extrude antibiotics, typical for pumps of that family. However it is inactivated in the presence of the acrAB pump which may extrude compounds preferentially over the emrAB pump and so the absence of the pump in that context might not reveal its ability to pump some or all of the substrates tested. Alternatively, HI1340 may be capable of functioning with the acrAB pump but may not be expressed significantly. The potential role of HI1340 as an efflux pump component remains to be determined.

### Example 3

### acrAB and Decreased Susceptibility to PDF Inhibitors in H. influenzae Clinical Strains

Decreased susceptibility to antibiotics in clinical isolates of a number of bacteria has been associated with over-expression of efflux pumps. Historically, identification of repressor mutations and/or pump gene over-expression in clinical isolates has been taken as sufficient to attribute decreased resistance to efflux, which is likely often the case, however there is not always a clear association between repressor gene mutations and/or pump expression status and resistance to specific antibiotics. See Sobel, McKay and Poole, Antimicrob Agents Chemother, Vol. 47, No. 10, pp. 3202-3207 (2003). Therefore to directly address whether the acrAB pump plays a significant role in mediating decreased susceptibility in those clinical strains with the lowest susceptibilities to the PDF inhibitors, acrB is inactivated (see FIG. 1) in strains NB65016, NB65027, NB65051, NB65063, NB65069 and NB65076, all of which exhibit decrease susceptibility to LBM415, LBK611 and clindamycin. Loss of the pump substantially increases susceptibility to both classes of antimicrobial in all cases, while having no impact on pump non-substrates (see Table 3 below) providing direct confirmation that the acrAB pump is widely-distributed and is a major contributor to decreased susceptibility exhibited by these strains. Moreover, inactivation of HI1462 (see FIG. 1), in strains NB65027 and NB65051, similarly increases susceptibility specifically to pump substrates (see Table 3), further confirming the role of this outer membrane channel in acrAB pump action in clinical isolates.

TABLE 3
Contribution of the acrAB Efflux Pump to Decrease Susceptibility
to LBM415 and LBK611 in Various H. influenzae Clinical Isolates
and Role of acrR in Modulating Resistance

| | MIC (μg/mL) | | | | | |
|---|---|---|---|---|---|---|
| Strain | Knockout | LBM415 | LBK611 | ERM | CLD | TET |
| NB65016 | | 16 | 16 | 16 | 16 | 1 |
| NB65016-CDS0004 | acrB | 0.25 | 0.25 | 0.25 | 0.5 | 1 |
| NB65027 | | 32 | 32 | 16 | 32 | 1 |
| NB65027-CDS0021 | acrB | 0.5 | 0.5 | 0.25 | 2 | 0.5 |
| NB65027-CDS0003 | HI1462 | 0.5 | 0.25 | 0.125 | 2 | 1 |
| NB65051 | | 32 | 32 | 16 | 32 | 1 |
| NB65051-CDS0002 | acrB | 0.25 | 0.25 | 0.25 | 0.5 | 1 |
| NB65051-CDS0022 | HI1462 | 0.25 | 0.25 | 0.125 | 0.5 | 1 |
| NB65063 | | 32 | 64 | 8 | 16 | 1 |
| NB65063-CDS0005 | acrB | 1 | 1 | 0.25 | 0.5 | 1 |
| NB65069 | | 16 | 32 | 16 | 16 | 1 |
| NB65069-CDS0007 | acrB | 0.25 | 0.5 | 0.125 | 0.5 | 1 |
| NB65076 | | 16 | 32 | 16 | 32 | 2 |
| NB65076-CDS0006 | acrB | 0.25 | 0.25 | 0.125 | 0.5 | 1 |
| NB65044 | | 4 | 4 | 4 | 8 | 0.5 |
| NB65044-CDS0008 | acrR | 8 | 16 | 8 | 32 | 0.5 |
| NB65044-CDS0011 | | 32 | 32 | 8 | 32 | 1 |
| NB65044-CDS0014 | | 32 | 32 | 8 | 32 | 1 |

ERM = erythromycin
CLD = clindamycin
TET = tetracycline
Note:
Strains NB65044-CDS0011 and NB65044-CDS0014 are acrR mutants selected on chocolate agar plates containing 8 μg/mL LBM415.
NB65044-CDS0011 acrR has a C → T nucleotide change at position 253, introducing a stop codon.
NB65044-CDS0014 acrR has a T → C nucleotide change at position 164 resulting in an L → P amino acid substitution.

### Example 4

### acrR is a Repressor of acrAB Pump Expression

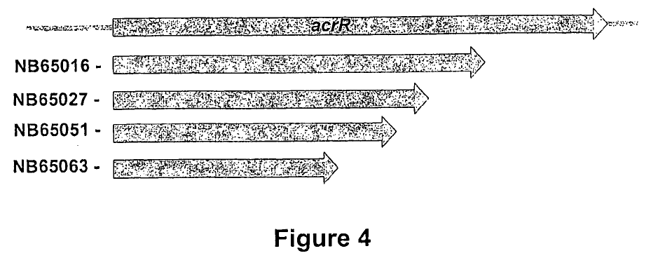

The demonstration that the acrAB efflux pump is the major contributor to decreased susceptibility to LBM415 and macrolides in H. influenzae clinical isolates with reduced susceptibility to these agents suggests that increased pump expression leads to decreases in susceptibility. Although the emerging picture of efflux pump regulation is becoming increasingly complex, there are many cases where pump over-expression is the result of simple mutations in regulatory genes. For example, P. aeruginosa nalB strains over-express mexAB-oprM due to mutations in the mexR gene encoding a repressor, located immediately upstream of the mexAB-oprM genes. See Poole et al., Antimicrob Agents Chemother, Vol. 40, No. 9, pp. 2021-2028 (1996). In H. influenzae, ORF HI0983, located immediately upstream of acrAB (see FIG. 1), encodes an acrR/tetR family repressor which may be involved in controlling expression of acrAB. Nucleotide sequencing of HI0893 from NB65016, NB65027, NB65051 and NB65063 reveals the presence of insertion/deletions or point mutations leading to either frameshifts or introduction of stop codons. See FIG. 4. Sequencing of the acrR genes from NB65069 and NB65076 reveals point mutations leading to amino acid changes relative to the published sequence for the acrR gene (data not shown). This preponderance of acrR mutations strongly suggests that the acrAB efflux pump is being over-expressed in these strains due to loss of acrR repressor function.

### Example 5

### Role of H. influenzae acrR in Modulating Susceptibility to LBM415 and Macrolides

To further examine whether acrR is a negative regulator of acrAB pump expression, and consequently if loss of acrR function results in decreased susceptibility to LBM415 and macrolides, the acrR gene is insertionally-inactivated in the laboratory strain NB65044. See FIG. 1. This results in a 2-fold decrease in susceptibility to LBM415 and a 4-fold decrease in susceptibility to LBK611 and clindamycin, while not altering susceptibility to the pump non-substrate tetracycline NB65044-CDS0008. See Table 3. This suggests that acrR is acting as a negative repressor of acrAB gene expression. However, the kanamycin cartridge resides in acrR in the same orientation as the downstream acrAB locus (see FIG. 1) and increased expression of acrAB resulting from polar effects cannot be ruled out. Therefore, to further clarify the role of acrR mutation in decreasing susceptibility to LBM415 and macrolides, it is tested whether exposure of NB65044 to LBM415 at 8 μg/mL will select mutants with altered acrR genes. Mutants of strain NB65044 are selected on chocolate agar containing 8 μg/mL of LBM415 (frequency; 10⁻⁶-10⁻⁹), and examination of the acrR genes from 10 isolated mutants reveals acrR mutations in all 10 isolates (data not shown).

Susceptibility testing of two of these mutants, NB65044-CDS0014 and NB65044-CDS0014, possessing an introduced stop codon and an amino acid change, respectively, (see Table 3 legend) reveals an 8-fold decrease in susceptibility to LBM415 and LBK611 and a 4-fold decrease in susceptibility to clindamycin with, again, no change in susceptibility to tetracycline. See Table 3. Furthermore, expression profiling reveals an increased abundance of acrAB transcripts (approximately 2.5-fold) in both mutants, as well as the acrR inactivated strain NB65044-CDs0011, supporting increased expression of the pump (data not shown). A similar increase in abundance of acrR transcripts is also observed suggesting autoregulation of acrR, a phenomenon observed for many efflux pump regulatory genes, such as mexZ and mexR in P. aeruginosa. The similarity in drug resistance profiles between the selected acrR mutants NB65044-CDs0011 and NB65044-CDS0014, and strain NB65044-CDS0008, where acrR is inactivated by insertion of a TN903 kanamycin resistance determinant in the same orientation as the downstream acrAB locus, suggests that over-expression of the pump in strain NB65044-CDS0008 does not result from polar effects from the TN903 cartridge promoter. Taken together, these data will show that decreased susceptibility to LBM415 can be acquired mutationally in the form of acrAB efflux pump over-expression resulting from acrR mutations. This contrasts previous reports of inactivation of acrR in H. influenzae, where no alteration of susceptibility to a wide range of compounds in the acrR knockout strain is observed. See Trepod and Mott (2004), supra. Increased pump expression does not always affect all pump substrates equally, particularly in the case of modest increases in expression of pumps, such as mexAB-oprM or acrAB, which are already expressed at significant levels. Since pump efficiency is likely specifically related to individual substrate properties, significant changes in susceptibility resulting from modest pump over-expression might only be observed for certain subsets of pump substrates that are efficiently extruded at typical levels of pump expression, including in this case, LBM415, LBK611 and clindamycin.

### Example 6

### Potentiation of LBM415 by PABN and Reserpine in H. influenzae and E. coli

H. influenzae NB65044-CDS0011 is used for H. influenzae testing. This strain has increased pump expression which is thought to result in more sensitive detection of pump inhibition.

For E. coli testing, strain NB27006 is used. This strain is deficient in its native AcrAB pump (described in H Okusu et al. J. Bacteriol. 1996 178: 306-308) and is a suitable host for cloning the H. influenzae acrAB pump genes.

H. influenzae: MIC determinations are conducted in 96 well sterile plates with a range of LBM415 along one axis and range of efflux inhibitor along the other axis. Medium used is HTM (Remel). Inoculum is set to approximately 10-5 bacteria per well using the BBL prompt system.

E. coli: MIC determines are conducted as described above except using Mueller-Hinton test medium.

Genes encoding H. influenzae AcrAB pump components are PCR amplified from NB65044 genomic DNA using the primers acrA prom F1 (5′-AATTACGTAAGATAA CCTAAGTGCG-3′) (SEQ ID NO. 11) and acrBR3 potentiation of LBM415 by MC-207,110 and Reserpine in H. influenzae and E. coli 5′-TATTAGCGGAATTATCTGAAG-3″) (SEQ ID NO. 12). The acrAB containing product is cloned into Topo PCR2.1 (Invitrogen) and transformed into Top10 competent cells (Invitrogen). An insert bearing plasmid is isolated and sequenced to ensure that mutations are not introduced during PCR and cloning. This plasmid is then transformed into E. coli strain NB27006, which lacks its native AcrAB-ToIC pump function.

Since LBM415 is known to be pumped by AcrAB-ToIC, potentiation of activity by MC-207,110 is consistent with the reported ability of this compound to interfere with RND family pump function. MC-207,110 has also been observed to potentiate clindamycin, another substrate that is extruded relatively efficiently by AcrAB-ToIC in H. influenzae. There is no potentiation of the non pump substrate tetracycline observed in the potentiation of LBM415 by MC-207, 110. Taken together, the data (not shown) suggest that the effect on LBM415 is indeed the result of the general ability of MC-207,110 to interfere with extrusion by the AcrAB-ToIC efflux pump. However, Reserpine did not potentiate either LBM415 or clindamycin (data not shown). Reserpine is a well characterized inhibitor of for example ABC transporter based efflux but has shown little activity against RND family pumps.

### Example 7

### MC-207,110 Activity Against the H. influenza AcrAB Efflux Components in E. coli

The activity of MC-207,110 in potentiation of two pump substrates, clindamycin and LBM415 while not impacting the pump non substrate tetracycline, in H. influenzae overexpressing the AcrAB pump components suggests that MC-207, 110 functions, weakly, as an AcrAB-ToIC pump inhibitor in H. influenzae. To further examine this, the H. influenzae acrAB genes are cloned and used to complement, in trans, an AcrAB pump deficiency in E. coli strain NB27006. The native AcrAB-ToIC pump of E. coli has been shown to confer significant intrinsic resistance to LBM415 (MIC typically 128 μg/ml and higher). Correspondingly, strains deficient in the AcrAB-ToIC pump function are sensitive (MIC typically approximately 1 μg/ml). Complementation of the pump defect in E. coli strain NB27006 using the H. influenzae acrAB genes results in significantly increased resistance to LBM415 (MIC 32 μg/ml). This indicates that the acrAB genes are functional in E. coli and that they are able to form a functional unit with the E. coli ToIC outer membrane channel.

Since efflux pumps are impacted by such factors as outer membrane permeability, and E. coli is thought to have a less permeable outer membrane than H. influenzae (possibly accounting for increased impact of efflux on drug resistance), it has reasoned that the detection of H. influenzae AcrAB pump inhibition might be more sensitive in an E. coli background. Consistent with this, the potentiating activity of MC-207,110 for both LBM415 and clindamycin in E. coli NB2007 complemented in trans with the H. influenzae acrAB genes is observed. Furthermore, no potentiation of tetracycline is detected. This is interesting in that the native E. coli AcrAB-ToIC pump does extrude tetracycline. This suggests that the antibiotic substrate profile of the H. influenzae pump is being preserved in the E. coli strain. This data further supports the notion that MC-207, 110 is able to interfere with extrusion of LBM415 by the H. influenzae AcrAB-ToIC pump.

We claim:

1. A method for treating a Gram-negative bacterial infection in a mammal comprising administering an effective amount of a compound of formula (I) 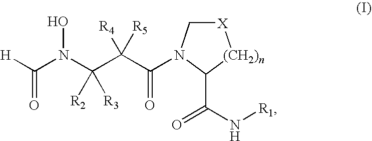
wherein
X is —CH₂—, —S—, —CH(OH)—, —CH(OR)—, —CH(SH)—, —CH(SR)—, —CF₂—, —C═N(OR)— or —CH(F)—, wherein R is alkyl;
R₁ is aryl or heteroaryl;
each of R₂, R₃, R₄ and R₅, independently, is hydrogen or alkyl, or
R₂ or R₃ and R₄ or R₅, collectively, form a C₄₋₇cycloalkyl; and
n is 0-3, provided that when n is 0, X is —CH₂—,
or a salt thereof or a prodrug thereof;
and an efflux pump inhibitor.

2. The method of claim 1, wherein the heteroaryl is a residue of formula (III) 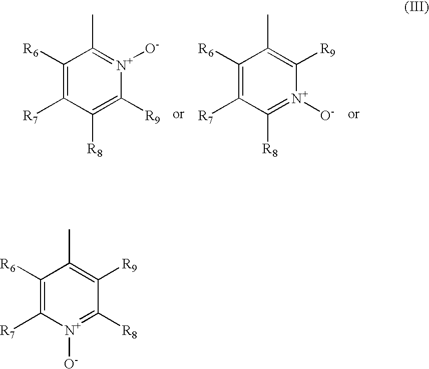
wherein
each of R₆, R₇, R₈ and R₉, independently, is hydrogen, alkyl, substituted alkyl, phenyl, halogen, hydroxy or alkoxy.

3. The method of claim 11, wherein the compound of formula (III) is

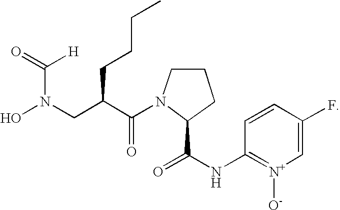

4. The method of claim 2, wherein the bacterial infection is caused by Gram-negative bacteria possessing an RND efflux pump.

5. The method of claim 4, wherein the Gram-negative bacteria is selected from the group consisting of E. coli, P. aeruginosa, H. influenzae and Neisseria gonorrhoeae.

6. The method of claim 5, wherein the Gram-negative bacteria is H. influenzae.

7. A method for treating a Gram-negative bacterial infection in a mammal which is caused by Gram-negative bacteria possessing an RND efflux pump, the method comprising administering an effective amount of the compound 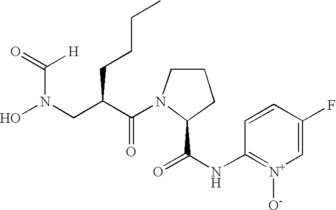
and an efflux inhibitor that inhibits the RND efflux pump in the Gram-negative bacteria.

8. The method of claim 7, wherein the Gram-negative bacteria is selected from the group consisting of E. coli, P. aeruginosa, H. influenzae and Neisseria gonorrhoeae.

9. The method of claim 8, wherein the Gram-negative bacteria is H. influenzae.

10. A method for increasing the susceptibility of Gram-negative bacteria to a compound of formula (I): 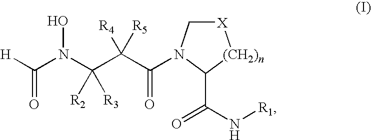
wherein
X is —CH₂—, —S—, —CH(OH)—, —CH(OR)—, —CH(SH)—, —CH(SR)—, —CF₂—, —C═N(OR)— or —CH(F)—, wherein R is alkyl;
R₁ is aryl or heteroaryl;
each of R₂, R₃, R₄ and R₅, independently, is hydrogen or alkyl, or
R₂ or R₃ and R₄ or R₅ collectively form a C₄₋₇cycloalkyl; and
n is 0-3, provided that when n is 0, X is —CH₂—; or
a salt thereof or a prodrug thereof, the method comprising:
contacting the bacteria with the compound of formula (I) and an efflux pump inhibitor in an amount effective to inhibit an efflux pump in the Gram-negative bacteria.

11. The method of claim 10, wherein the heteroaryl is a residue of formula (III) 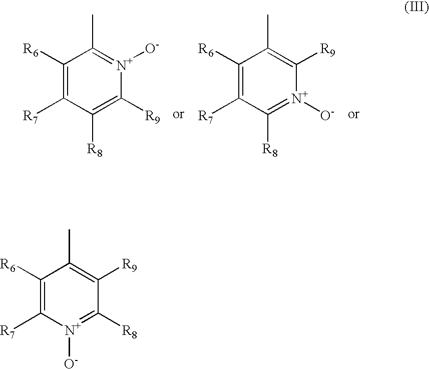
wherein
each of R₆, R₇, R₈ and R₉, independently, is hydrogen, alkyl, substituted alkyl, phenyl, halogen, hydroxy or alkoxy.

12. The method of claim 11, wherein the compound of formula (III) is

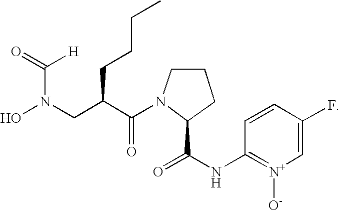

13. The method of claim 10, wherein the efflux pump in the Gram-negative bacteria is an RND efflux pump.

14. The method of claim 13, wherein the Gram-negative bacteria is selected from the group consisting of E. coli, P. aeruginosa, H. influenzae and Neisseria gonorrhoeae.

15. The method of claim 14, wherein the Gram-negative bacteria is H. influenzae.

16. A method for increasing the susceptibility of Gram-negative bacteria possessing an RND efflux pump to the compound 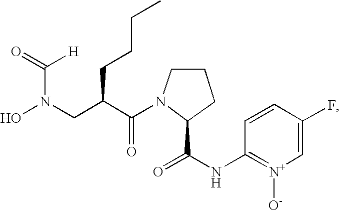
comprising contacting the Gram-negative bacteria with the compound and an efflux pump inhibitor in an amount effective to inhibit the RND pump in the Gram-negative bacteria.

17. The method of claim 16, wherein the Gram-negative bacteria is selected from the group consisting of E. coli, P. aeruginosa, H. influenzae and Neisseria gonorrhoeae.

18. The method of claim 17, wherein the Gram-negative bacteria is H. influenzae.

19. A pharmaceutical composition comprising a compound of formula (I) 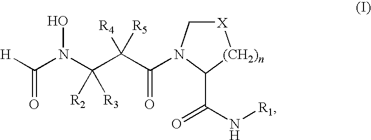
wherein
X is —CH₂—, —S—, —CH(OH)—, —CH(OR)—, —CH(SH)—, —CH(SR)—, —CF₂—, —C═N(OR)— or —CH(F)—, wherein R is alkyl;
R₁ is aryl or heteroaryl;
each of R₂, R₃, R₄ and R₅, independently, is hydrogen or alkyl, or
R₂ or R₃ and R₄ or R₅, collectively, form a C₄₋₇cycloalkyl; and
N is 0-3, provided that when n is 0, x is —CH₂—,
or a salt thereof or a prodrug thereof, an efflux pump inhibitor and a pharmaceutically acceptable carrier.

20. The pharmaceutical composition of claim 19, wherein the heteroaryl of the compound of formula (I) is a residue of formula (III) 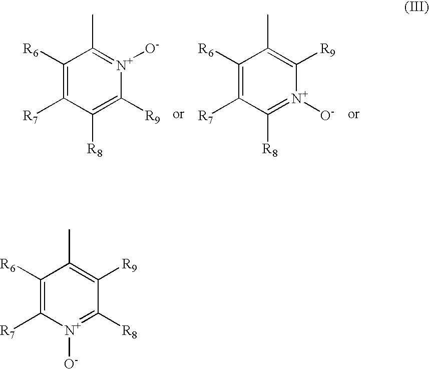
wherein
each of R₆, R₇, R₈ and R₉, independently, is hydrogen, alkyl, substituted alkyl, phenyl, halogen, hydroxy or alkoxy.

21. The pharmaceutical composition of claim 20, wherein the compound of formula (III) is

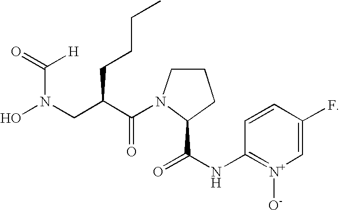

22. The pharmaceutical composition of claim 19, wherein the efflux inhibitor inhibits an RND efflux pump in Gram-negative bacteria.

23. The pharmaceutical composition of claim 22, wherein the Gram-negative bacteria is selected from the group consisting of E. coli, P. aeruginosa, H. influenzae and Neisseria gonorrhoeae.

24. The pharmaceutical composition of claim 23, wherein the Gram-negative bacteria is H. influenzae.


SEQUENCE LISTING (12 sequences)

SEQ ID NO: 1 - DNA, 25 bp, Artificial Sequence
  Description of Artificial Sequence Synthetic primer
ccactttaatgtatgaggaaatccg

SEQ ID NO: 2 - DNA, 27 bp, Artificial Sequence
  Description of Artificial Sequence Synthetic primer
cgaattacgtaagataacctaagtgcg

SEQ ID NO: 3 - DNA, 25 bp, Artificial Sequence
  Description of Artificial Sequence Synthetic primer
gccacgttgtgtctcaaaatctctg

SEQ ID NO: 4 - DNA, 24 bp, Artificial Sequence
  Description of Artificial Sequence Synthetic primer
ccagtgttacaaccaattaaccaa

SEQ ID NO: 5 - DNA, 27 bp, Artificial Sequence
  Description of Artificial Sequence Synthetic primer
cacgcttgctttgttgatgtctggtgc

SEQ ID NO: 6 - DNA, 25 bp, Artificial Sequence
  Description of Artificial Sequence Synthetic primer
tcccgccattgagctatataccgca

SEQ ID NO: 7 - DNA, 24 bp, Artificial Sequence
  Description of Artificial Sequence Synthetic primer
taatgatgaaaagtgcggttaatt

SEQ ID NO: 8 - DNA, 25 bp, Artificial Sequence
  Description of Artificial Sequence Synthetic primer
tttctgaatcgcacgccaagagcgt

SEQ ID NO: 9 - DNA, 24 bp, Artificial Sequence
  Description of Artificial Sequence Synthetic primer
gtgcggtgccaccgcaaggacata

SEQ ID NO: 10 - DNA, 25 bp, Artificial Sequence
  Description of Artificial Sequence Synthetic primer
tgcaggctctattgcacccacaatg

SEQ ID NO: 11 - DNA, 25 bp, Artificial Sequence
  Description of Artificial Sequence Synthetic primer
aattacgtaagataacctaagtgcg

SEQ ID NO: 12 - DNA, 21 bp, Artificial Sequence
  Description of Artificial Sequence Synthetic primer
tattagcggaattatctgaag